(19) United States
(12) Patent Application Publication
(10) Pub. No.: US 2026/0345987 A1
(43) Pub. Date: Nov. 5, 2026

(54) Co-Molded Rigid Syringe Cap With Integrated RFID and Torque Transmission Features

(71) Applicant: Becton Dickinson France, Le Pont-de-Claix (FR)

(72) Inventors: Simon Rempfer, St Hilaire de la Cote (FR); Senturk Ozturk, Echirolles (FR); Lukasz Dolega, St Martin Le Vinoux (FR); Jérémy Zucchelli, St Martin d'Heres (FR); Jérémie Vaxelaire, Ambilly (FR)

(21) Appl. No.: 19/484,021

(22) PCT Filed: May 15, 2024
(86) PCT No.: PCT/EP2024/063414

§ 371 Date: Nov. 13, 2025

(30) Foreign Application Priority Data
May 30, 2023 (EP) 23305856.9

(51) Int. Cl. A61M 39/20; A61M 5/178

(52) U.S. Cl. CPC A61M 39/20; A61M 5/178; A61M 2205/0238; A61M 2205/586; A61M 2205/60

(57) ABSTRACT

Provided herein is a syringe cap including an inner molded portion having a first end and a second end, with the first end being an open end and including a female connector formed thereon and the second end being a closed end. The syringe cap also includes an RFID element positioned over the inner molded portion at the second end. The syringe cap further includes an outer molded portion formed over the inner molded portion and over the RFID element at the second end, so as to encase the RFID element between the inner and outer molded portions, with the outer molded portion having an outer surface including a plurality of spline teeth spaced circumferentially thereabout and extending radially outward from the outer surface, wherein the spline teeth have a height sufficient to enable a torque transfer from a twisting tool to the syringe cap during cap fastening.

### CROSS-REFERENCE TO RELATED APPLICATION

The present application claims priority to European Application No. 23305856.9 entitled “Co-Molded Rigid Syringe Cap with Integrated RFID and Torque Transmission Features” filed May 30, 2023, the entire disclosure of which is hereby incorporated by reference.

### BACKGROUND OF THE INVENTION

### Field of the Invention

The present disclosure relates generally to syringe caps and, more particularly, to syringe caps with an integrated RFID tag and torque transmission features formed thereon.

### Description of Related Art

Syringes are used in a variety of environments for administering liquids, such as medications or other drugs, to a patient. Many syringes are provided as prefilled syringes, which provide the convenience of rapidly delivering the liquid therein to a patient without the need to first aspirate the medication from another container and meter its volume. For dispensing fluids, the prefilled syringe will typically include a syringe barrel with an opening at a distal end adapted for fluid communication with a patient (e.g., a luer-type fitting (needleless access device) fitted at the distal end of the syringe barrel and connected to a fluid line of a patient) and a plunger assembly inserted through the opposite proximal end of the barrel. The plunger assembly typically includes an elongated plunger rod extending out of the barrel, and a plunger head or stopper disposed at the distal end of the plunger rod. The stopper or plunger stopper is typically made of elastomeric material, and comprises a body with a tail disposed at its proximal end adapted for attachment to the distal end of the plunger rod, and a head disposed at its distal end. On an external cylindrical wall of the body is defined a plurality of annular outwardly protruding ribs adapted to ensure the container closure integrity of a syringe when the stopper is inserted into the syringe.

In using prefilled syringes, it is desirable to inlay the prefilled syringe to include data concerning the syringe and/or the contents (i.e., medication) included in the syringe and, in some embodiments, to enable tracking of the syringe. That is, it is desired to have the ability to track the history of individual syringes or lots of syringes from early in the manufacturing life through to the end use of the device. Identification and/or tracking of the syringe may be provided via use of an electronic element that contains data thereon and that can be conventionally read in and read out in a contactless fashion. As one example, an electronic element such as, for example, an RFID tag, may be incorporated into the syringe to provide identification and tracking for the syringe. In some known syringes, the RFID tag is incorporated into a rigid syringe cap that is positioned over the opening of the syringe barrel on the distal end, such as a cap that mates with a luer-type fitting at the distal end of the syringe barrel. The RFID tag may be embedded within the syringe cap to provide protection thereto, with such embedding of the cap achieved via a co-molding process for forming the syringe cap.

While it is known to integrate an RFID tag into a syringe cap to provide identification and/or tracking of a syringe, existing syringe caps with integrated RFID tags may suffer from issues associated with the functionality of the syringe cap. As one example, it is known that batches of syringes are typically packaged, stored, and shipped in storage containers known as “nests,” and that the sizing/diameter of the syringe cap may need to be controlled in order to enable machine automated placement and/or withdrawal of syringes into and/or out from the nest without interference. However, the integration of an RFID tag within a syringe cap via a two-step molding process can lead to challenges in controlling the cap diameter, while still achieving desired functionality with the cap. Specifically, it is desired to include splines (“spline teeth”) on the outer surface of the syringe cap which by the cap can be gripped and twisted via an associated twisting tool in order to secure the cap onto the syringe (i.e., to engage a female luer connection on the cap with a male luer connection on the syringe barrel), but inclusion of the RFID tag can reduce the space available for such spline teeth to be formed on the syringe cap. Accordingly, the height of the spline teeth is reduced in order to keep the syringe cap below a required dimensional limit, and this reduction in the spline height and the amount they protrude out from an adjacent cap outer surface can have a negative impact on external torque transmission from a twisting tool to the syringe cap when securing the cap to the syringe. As an example, the spline teeth height on some existing caps is 0.30 mm, and this height of the spline teeth can have a negative impact on external torque transmission from a twisting tool to the syringe cap when securing the cap to the syringe.

As another example, it is recognized that inclusion of the RFID tag between the two molded members of the syringe cap can lead to adhesion issues in the cap. That is, due to the material difference present between the first molded member and a first surface of the RFID tag and between the second molded member and a second surface of the RFID tag, the integrity of the syringe cap may become comprised upon application of an external torque to the cap—with a decohesion occurring between the molded members and the RFID tag. This decohesion may result in a reduction of the torque transferred from a twisting tool and through the syringe cap (i.e., an internal torque transmission), to mate the cap with the syringe barrel.

Accordingly, a need exists in the art for a syringe cap that incorporates an RFID tag therein but that includes features thereon that provide for efficient external and internal torque transmission.

### SUMMARY OF THE INVENTION

Provided herein is a syringe cap for use with a syringe. The syringe cap includes an inner molded portion having a first end and a second end and defining an internal cavity, with the first end being an open end and including a female connector formed thereon and the second end being a closed end. The syringe cap also includes a radio frequency identification (RFID) element positioned over the inner molded portion at the second end, so as to extend circumferentially about at least a portion of the second end. The syringe cap further includes an outer molded portion formed over the inner molded portion and over the RFID element at the second end, so as to encase the RFID element between the inner molded portion and the outer molded portion, with the outer molded portion having an outer surface including a plurality of spline teeth spaced circumferentially thereabout and extending radially outward from the outer surface, wherein the plurality of spline teeth have a height of 0.400 mm or more extending radially out from the outer surface.

In certain configurations, each of the plurality of spline teeth extend out between 0.400 and 0.750 mm from the outer surface.

In certain configurations, the second end of the inner molded portion comprises a circumferential outer surface, and wherein an outer diameter of the second end is approximately 9.75 mm.

In certain configurations, the female connector has a threaded outer surface configured to engage a male connector, and the height of the plurality of spline teeth enables engagement of the second end with a twisting tool, for threadingly engaging the threaded outer surface of the female connector to the male connector.

In certain configurations, the inner molded portion comprises an annular flange formed distally from the threaded outer surface, between the first end and the second end.

In certain configurations, the RFID element comprises a flat RFID inlay that conforms to a cylindrical outer surface of the first end of the inner molded portion.

In certain configurations, the second end of the inner molded portion comprises a rib formed on a cylindrical outer surface thereof, the rib extending radially outward from the cylindrical outer surface.

In certain configurations, the rib comprises an angular locator, which the RFID element is positioned relative to, with the RFID element positioned over the cylindrical outer surface of the second end so as to not overlap the rib.

In certain configurations, the outer molded portion is formed over the inner molded portion, including over the rib, with the rib forming an interference between the inner molded portion and the outer molded portion.

In certain configurations, a radially outward facing surface of the rib has a concave shape.

In certain configurations, the outer molded portion is formed over the inner molded portion, except for the rib, such that the rib is not covered by the outer molded portion, and wherein a radially outward facing surface of the rib is flush with the outer surface of outer molded portion.

In certain configurations, the outer molded portion is formed over the inner molded portion, except for the rib, such that the rib is not covered by the outer molded portion, and wherein a portion of rib extends radially outward past the outer surface of the outer molded portion to a height equal to the height of the plurality of spline teeth.

Also provided herein is a syringe that includes a syringe barrel having a proximal end and a distal end and defining a chamber, with the syringe barrel having an opening at the proximal end. The syringe also includes a plunger assembly inserted through the opening and axially movable within the chamber of the syringe barrel, with the plunger assembly including a plunger rod having an elongated body and extending between a proximal end and a distal end, and a stopper attached to the distal end of plunger rod and positioned within the barrel chamber. The syringe further includes a syringe cap having an inner molded portion having a first end and a second end and defining an internal cavity, with the first end being an open end and including a female connector formed thereon and the second end being a closed end. The syringe cap also includes a radio frequency identification (RFID) element positioned over the inner molded portion at the second end, so as to extend circumferentially about at least a portion of the second end. The syringe cap further includes an outer molded portion formed over the inner molded portion and over the RFID element at the second end, so as to encase the RFID element between the inner molded portion and the outer molded portion, with the outer molded portion having an outer surface including a plurality of spline teeth spaced circumferentially thereabout and extending radially outward from the outer surface. The female connector of the inner molded portion is coupled to a male connector provided at the distal end of the syringe barrel.

Also provided herein is a method of forming a co-molded syringe cap. The method includes molding an inner cap portion having a first end and a second end and defining an internal cavity, with the first end being an open end that includes a female connector formed thereon and the second end being a closed end. The method also includes setting a radio frequency identification (RFID) element onto the inner molded portion at the second end, with the RFID element set so as to extend circumferentially about at least a portion of the second end. The method further includes molding an outer cap portion over the inner molded portion and over the RFID element at the second end, so as to encase the RFID element between the inner cap portion and the outer cap portion, the outer cap portion including an outer surface having a plurality of spline teeth spaced circumferentially thereabout and extending radially outward from the outer surface. The plurality of spline teeth are formed to have a height of 0.400 mm or more extending radially out from the outer surface.

In certain configurations, the outer cap portion is molded such that each of the plurality of spline teeth is formed to extend out between 0.400 and 0.750 mm from the outer surface.

In certain configurations, the inner cap portion is molded such that the second end of the inner molded portion is formed to have a circumferential outer surface, with an outer diameter of the second end being approximately 9.75 mm.

In certain configurations, the inner cap portion is molded to form a rib on a cylindrical outer surface of the inner cap portion, the rib extending radially outward from the cylindrical outer surface.

In certain configurations, the outer molded portion is molded over the inner molded portion, including over the rib, with the rib forming an interference between the inner molded portion and the outer molded portion.

In certain configurations, the outer molded portion is molded over the inner molded portion, except for the rib, such that the rib is not covered by the outer molded portion, with a radially outward facing surface of the rib being flush with the outer surface of outer molded portion or extending radially outward past the outer surface of the outer molded portion to a height equal to the height of the plurality of spline teeth.

In certain configurations, the RFID element is set over the cylindrical outer surface of the second end of the inner molded portion so as to not overlap the rib, with the rib being an angular locator relative to which the RFID element is positioned.

Also provided herein is a syringe cap for use with a syringe. The syringe cap includes an inner molded portion having a first end and a second end and defining an internal cavity, with the first end being an open end and including a female connector formed thereon and the second end being a closed end. The syringe cap also includes a radio frequency identification (RFID) element positioned over the inner molded portion at the second end, so as to extend circumferentially about at least a portion of the second end. The syringe cap further includes an outer molded portion formed over the inner molded portion and over the RFID element at the second end, so as to encase the RFID element between the inner molded portion and the outer molded portion, with the outer molded portion having an outer surface including a plurality of spline teeth spaced circumferentially thereabout and extending radially outward from the outer surface. The second end of the inner molded portion includes a rib formed on a cylindrical outer surface thereof, the rib extending radially outward from the cylindrical outer surface, to form an interference between the inner molded portion and the outer molded portion.

In certain configurations, the rib comprises an angular locator, which the RFID element is positioned relative to, with the RFID element positioned over the cylindrical outer surface of the second end so as to not overlap the rib.

In certain configurations, the outer molded portion is formed over the inner molded portion, including over the rib.

In certain configurations, the outer molded portion is formed over the inner molded portion, except for the rib, such that the rib is not covered by the outer molded portion, and wherein a radially outward facing surface of the rib is flush with the outer surface of outer molded portion.

In certain configurations, the outer molded portion is formed over the inner molded portion, except for the rib, such that the rib is not covered by the outer molded portion, and wherein a portion of rib extends radially outward past the outer surface of the outer molded portion to a height equal to the height of the plurality of spline teeth.

### BRIEF DESCRIPTION OF THE DRAWINGS

FIG. 1 is a perspective view of a syringe, with which embodiments of the disclosure may be implemented;

FIG. 2 is an exploded view of the syringe of FIG. 1;

FIG. 3 is a perspective view of a syringe cap included in syringe of FIG. 1, with an end wall of the cap removed, according to a non-limiting embodiment described herein;

FIG. 4 is a side cross-sectional view of the syringe cap of FIG. 3, shown coupled to a male connector of a syringe;

FIG. 5 is a perspective view of an inner molded portion of the syringe cap of FIG. 3, with an RFID tag positioned thereon;

FIG. 6 is a perspective view of a syringe cap included in syringe of FIG. 1, with an end wall of the cap removed, according to another non-limiting embodiment described herein;

FIG. 7 is a perspective view of an inner molded portion of the syringe cap of FIG. 6, with an RFID tag positioned thereon;

FIG. 8 is a perspective view of a syringe cap included in syringe of FIG. 1, with an end wall of the cap removed, according to another non-limiting embodiment described herein;

FIG. 9 is a perspective view of an inner molded portion of the syringe cap of FIG. 8, with an RFID tag positioned thereon;

FIG. 10 is a perspective view of a syringe cap included in syringe of FIG. 1, with an end wall of the cap removed, according to another non-limiting embodiment described herein;

FIG. 11 is a perspective view of an inner molded portion of the syringe cap of FIG. 10, with an RFID tag positioned thereon;

FIG. 12 is a perspective view of a syringe cap included in syringe of FIG. 1, with an end wall of the cap removed, according to another non-limiting embodiment described herein; and

FIG. 13 is a perspective view of an inner molded portion of the syringe cap of FIG. 12, with an RFID tag positioned thereon.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a syringe in preparation for or during use. Similarly, in this application, the terms “in the distal direction” and “distally” mean in the direction toward the distal tip of the syringe, and the terms “in the proximal direction” and “proximally” mean in the direction opposite the direction of the distal tip of the syringe.

Aspects and embodiments of the disclosure are directed to a syringe cap with an integrated RFID tag and torque transmission features formed thereon. The syringe cap is formed as a co-molded cap with the RFID tag embedded therein between distinct molded portions or layers of the cap. The syringe cap includes internal and external features thereon that enable torque applied to the cap, such as via a process twisting or screwing tool engageable with the cap, to be transmitted through the cap, so as to provide for twisting engagement of the cap with an associated connection on a syringe barrel.

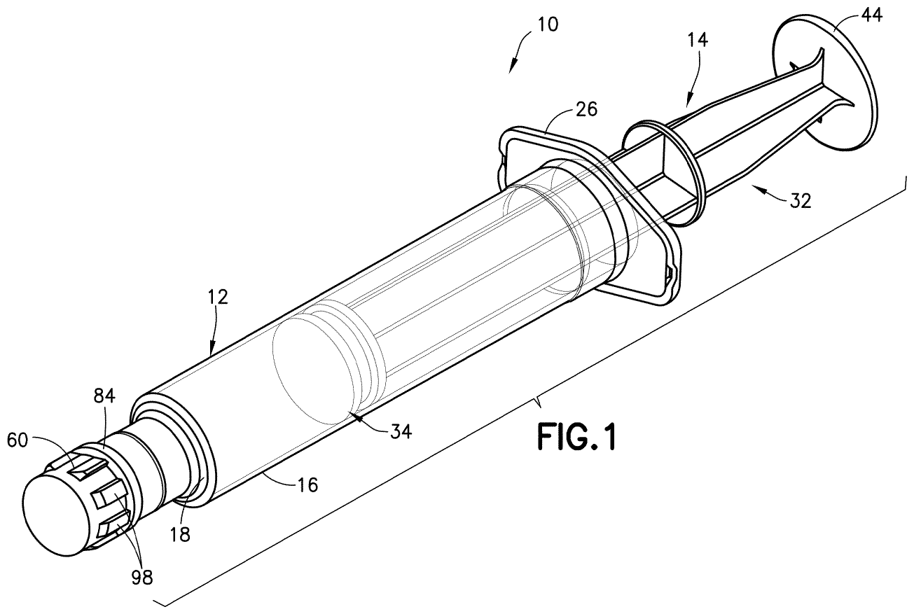

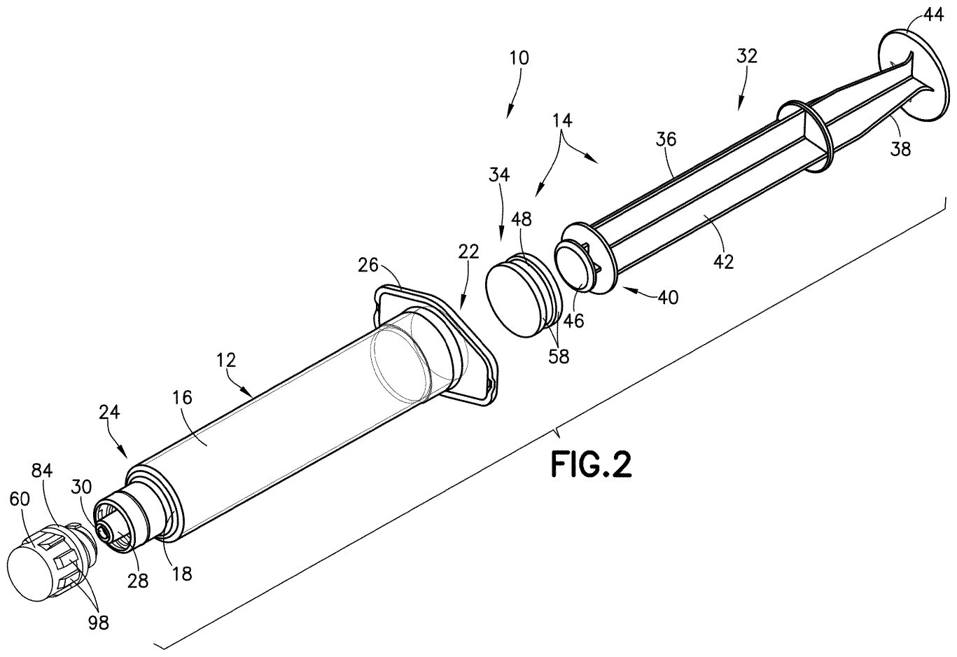

Referring to FIGS. 1 and 2, shown is a non-limiting embodiment of a syringe 10, such as a prefilled syringe for example, with which aspects or embodiments of the disclosure may be implemented. The syringe 10 generally includes a syringe barrel 12 and a plunger assembly 14. The plunger assembly 14 is axially movable within the syringe barrel 12 to an advanced position to facilitate administering of an injectable fluid (e.g., medication) to a patient, for example.

The syringe barrel 12 is formed of a generally cylindrical outer wall 16 and an end wall 18 that collectively define a chamber 20 for retaining fluid therein. The syringe barrel 12 includes an open proximal end 22 configured to receive the plunger assembly 14 therein and a distal end 24 at which end wall 18 is positioned. The proximal end 22 of the syringe barrel 12 may include a flange 26 to facilitate handling and positioning of the syringe 10 and to maintain the relative position of the syringe barrel 12 with respect to the plunger assembly 14 during medication administration. At the distal end 24, a nozzle 28 extends distally outward from the end wall 18 and defines a lumen 30 in fluid communication with the chamber 20. In some embodiments, the nozzle 28 may form part of a luer connection (i.e., male luer connection) that is coupleable to a corresponding needle-free connector (not shown) to which the syringe 10 is to be brought into engagement for administering medication to a patient. It can be appreciated that other types of attachment arrangements can be provided for securing the distal end 24 of the syringe barrel 12 for expelling the injectable material into a target location.

The plunger assembly 14 of syringe 10 is formed of an elongate plunger rod 32 (more generally “plunger 32,” as used hereafter) and a plunger head or stopper 34. The plunger 32 may include a main body 36 extending between a plunger proximal end 38 and a plunger distal end 40. In some embodiments, the main body 36 may include a plurality of elongate vanes or walls 42 extending axially along a length thereof between the plunger proximal end 38 and the plunger distal end 40. A thumb press 44 is positioned at the plunger proximal end 38 that may be engaged by a thumb (or other finger) of the user to apply a distally directed force to the plunger assembly 14 to move the plunger 32 with respect to the syringe barrel 12. In some embodiments, a flanged extension member 46 (e.g., disc-shaped flange) is positioned at the plunger distal end 40 that is configured to mate with the stopper 34. In other embodiments, the plunger distal end 40 may include a female receptacle formed therein that is configured to receive and connect to a protrusion (e.g., pin) extending out proximally from the stopper, with the protrusion and receptacle engaging via threading, for example.

The stopper 34 of plunger assembly 14 is positioned at the plunger distal end 40 so as to be movable along with the plunger 32 within the chamber 20 of syringe barrel 12. The stopper 34 may be made from a material that is different from the material of the plunger 32 and that is capable of forming a tight seal with the syringe barrel 12 as it is advanced therethrough. In some embodiments, the stopper 34 includes a receptacle (not shown) formed therein that is sized and configured to receive the flanged extension member 46 of plunger 32, with the flanged extension member 46 coupling with the receptacle via a press fit connection, for example, to secure the stopper 34 to the plunger 32, although it can be appreciated that the attachment member 48 and plunger distal end 40 can be secured by other techniques known in the art. The attachment member 48 may further include a pair of spaced-apart annular flanges 58 formed thereon (i.e., O-rings) that form a tight seal with the cylindrical outer wall 16 of syringe barrel 12.

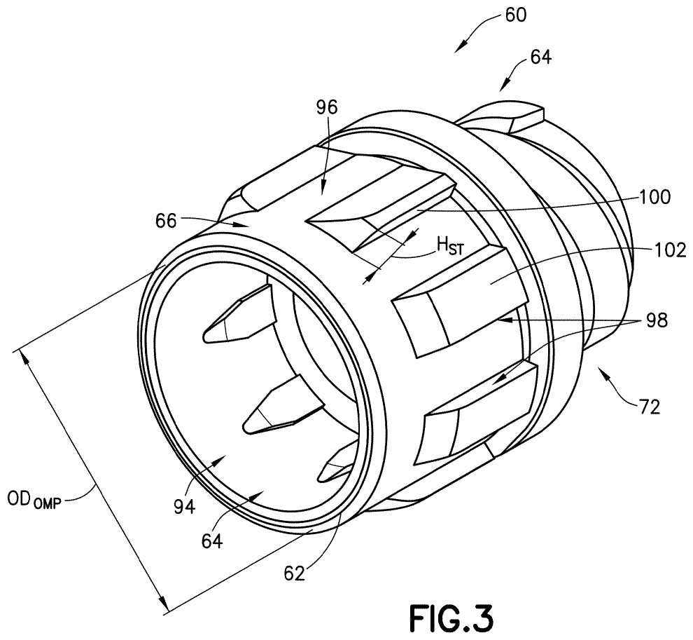

As shown in FIGS. 1 and 2 and now also in FIGS. 3-5, syringe 10 further includes a syringe cap 60 that may be coupled to the nozzle 28 of syringe barrel 12 to protect the nozzle 28 from contamination and/or maintain the chamber 20 in a sealed condition or state prior to use of the syringe 10. The syringe cap 60 may be configured as a rigid cap that is positioned about the nozzle 28, so as to provide a fluid-tight seal over the nozzle 28. According to aspects and embodiments of the disclosure, and as will be explained in further detail below, the syringe cap 60 includes an RFID element 62 integrated therein that allows for identification and/or tracking of the syringe 10. That is, the RFID element 62 may include or store information thereon regarding the contents of the syringe 10 (i.e., the medication or drug included therein, if a prefilled syringe) and/or may provide location information to an associated reader, so as to enable tracking of the syringe 10.

The syringe cap 60 is shown in further detail in FIGS. 3-5 according to one aspect or embodiment of the disclosure. The syringe cap 60 is formed as a rigid, co-molded cap that is formed via two separate molding processes—with an inner molded portion 64 (“inner cap portion”) formed during a first molding shot or process and an outer molded portion 66 (“outer cap portion”) formed during a second molding shot or process. Each of the inner and outer molded portions 64, 66 is thus formed of a material suitable for molding, such as a polymeric material that may be injection molded to produce the resulting inner and outer molded portions 64, 66. According to some aspects or embodiments, the polymeric material from which the inner and/or outer molded portions 64, 66 is formed may comprise a transparent or translucent material. As explained further below, in manufacturing of the syringe cap 60, the outer molded portion 66 is formed over part of the inner molded portion 64, with the RFID element 62 embedded between the inner and outer molded portions 64, 66.

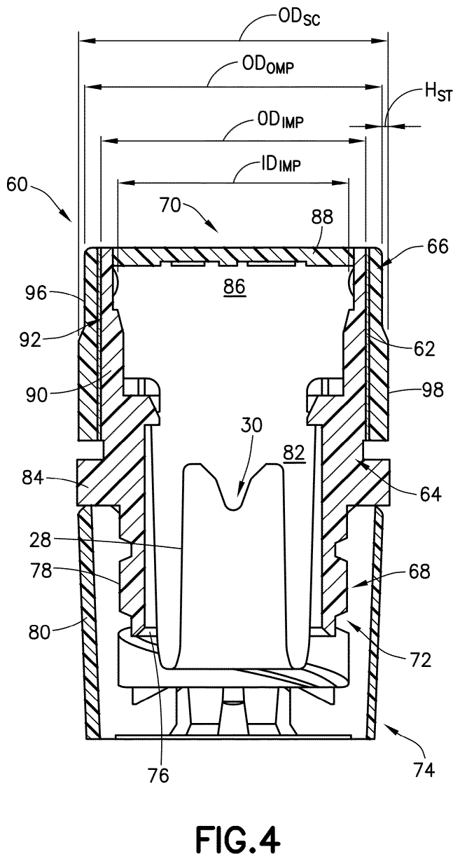

As best shown in FIGS. 4 and 5, the inner molded portion 64 of syringe cap 60 may generally be defined as including a first end 68 and a second end 70. The first end 68 is configured as an open end and includes a connector 72 formed thereon suitable for coupling to the syringe barrel 12. As previously described, in some embodiments the distal end of the syringe barrel 12 includes a nozzle 28 thereon that forms part of a male (luer) connector 74 and, in such embodiments, the connector 72 on the first end 68 of inner molded portion 64 may be configured as a female (luer) connector (hereafter “female connector 72”) configured to mate with the male connector 74, as shown in FIG. 4. The female connector 72 may include a cylindrical portion 76 having a threaded outer surface 78 configured to engage an inner surface of a threaded collar 80 of the male connector 74, with the cylindrical portion 76 defining an opening 82 within which the tapered nozzle 28 of the male connector 74 may be received.

Adjacent the threaded outer surface 78 of the female connector 72, an annular flange 84 may be formed on the inner molded portion 64. The annular flange 84 is positioned distally from the first end 68 of inner molded portion 64 (and between the first end 68 and the second end 70) such that, upon engagement of the threaded outer surface 78 with the threaded collar 80 of the male connector 74, the annular flange 84 will abut an end of the male connector 74, thereby providing a sealed engagement between the syringe cap 60 and the syringe barrel 12.

The second end 70 of inner molded portion 64 is configured as a closed end that, along with first end 68, defines a cavity 86 within the syringe cap 60. The second end 70 is formed of an end wall 88 and a continuous sidewall 90 that collectively provide the closed end of inner molded portion 64. The sidewall 90 provides a generally cylindrical outer surface 92 on the second end 70, while an inner circumferential surface 94 of the sidewall 90 defines part of the cavity 86 within inner molded portion 64.

The RFID element 62 of syringe cap 60 is applied or set onto the outer surface 92 of the sidewall 90 at the second end 70 of inner molded portion 64. The RFID element 62 may be provided in the form of a generally flat, two-dimensional RFID tag or inlay (hereafter “RFID tag 62”). While not shown, it is recognized that the RFID tag 62 may include an RFID antenna that is arranged on an antenna foil and on which a chip module with a carrier foil is fixed—with the chip module preferably situated between the two foils. According to embodiments, the RFID tag 62 conforms to the cylindrical outer surface 92 of the sidewall 90 and may wrap at least substantially thereabout.

As shown in FIGS. 3 and 4, after molding of inner molded portion 64 and application of the RFID tag 62 onto the second end 70 of the inner molded portion 64, the outer molded portion 66 may be formed (i.e., molded) over the inner molded portion 64 and over the RFID tag 62 to complete fabrication of the syringe cap 60. The outer molded portion 66 is formed over the inner molded portion 64 and over the RFID tag 62 at the second end 70 of the inner molded portion 64, so as to encase the RFID tag 62 between the inner molded portion 64 and the outer molded portion 66. The outer molded portion 66 is formed as a generally cylindrical, tube-shaped member that surrounds the second end 70 of inner molded portion 64. The outer molded portion 66 includes an outer surface 96, and a plurality of spline teeth 98 are formed on the outer surface 96. The spline teeth 98 are spaced circumferentially about the outer surface 96 of outer molded portion 66, with some embodiments having the spline teeth 98 spaced equidistantly from one another. Each of the spline teeth 98 extends radially outward from the outer surface 96 and, in some embodiments, at least some of the spline teeth 98 are configured such that at least one sidewall 100 thereof (extending between outer surface 96 and a top or radially outward facing surface 102 of the spline tooth 98) is orthogonal to the outer surface 96. This orthogonally oriented sidewall 100 of a spline tooth 98 thus presents a surface with which a twisting tool (i.e., process screwing tool) may engage to apply a screwing torque to the syringe cap 60 when securing the cap 60 to the syringe barrel 12—i.e., when engaging the threaded outer surface 78 of the female connector 72 on inner molded portion 64 with the threaded collar 80 of male connector 74 on syringe barrel 12.

In order to provide for proper engagement between a twisting tool and the spline teeth 98 and enable a desired transfer of screwing torque from the twisting tool to the spline teeth 98, it is desired for each of the spline teeth 98 to have a height H_{ST} greater than a minimum height that would negatively affect torque transfer. As previously indicated, some prior art syringe caps include spline teeth with a height of 0.300 mm, and this height is insufficient for enabling a desired transfer of screwing torque from the twisting tool to the spline teeth. According to aspects of the disclosure, it is desirable for the spline teeth 98 to have a height H_{ST} of 0.400 mm or greater, with it recognized that a maximum height H_{ST} of the spline teeth 98 is limited by a maximum overall diameter of the syringe cap 60 (e.g., 10.9 mm), as explained in more detail below. Thus, in some embodiments, the height H_{ST} of the spline teeth 98 is between 0.400 mm and 0.7500 mm, with spline teeth 98 having a height H_{ST} of 0.575 mm being a preferred example. Put another way, it is desired for the spline teeth 98 to have a height H_{ST} that is 133% to 250% of the spline tooth height of known syringe caps, as indicated by the spline tooth measurements provided above.

In accordance with aspects of the disclosure, it is recognized that an outer diameter of the syringe cap 60, outer diameter OD_{SC}, may be constrained based on packaging requirements of the syringe 10. That is, it is known that batches of syringes 10 are typically packaged, stored, and/or shipped in nests having numerous chimneys formed therein to hold the syringes. Removal of the syringes 10 from this nest is performed by axially sliding the syringes 10 relative to the nest and, if a syringe cap 60 exceeds a pre-determined diameter, the syringe 10 may become lodged within a chimney during extraction from the nest. Consequently, an automated processing line associated with the syringes 10, such as for automatically filling the syringes 10 with medication/drugs for example, may need to be stopped until the syringe(s) 10 are able to be dislodged from the nest, leading to undesirable downtime and maintenance costs, as well as potential damage to the syringe(s) 10.

In order to fabricate syringe caps 60 in accordance with the diameter constraints described above while still maintaining a desired spline tooth height H_{ST} of between 0.400 mm and 0.750 mm, aspects of the disclosure provide for performing the molding of the inner molded portion 64 in a manner that controls an outer diameter thereof (i.e., of the outer surface 92 of inner molded portion)—referred to as the inner molded portion outer diameter, OD_{IMP}. That is, it is recognized that the inner molded portion 64 and outer molded portion 66 of syringe cap 60 should have an appropriate thickness that provides for embedding of the RFID tag 62 therebetween and maintaining integrity of the syringe cap 60, but if an outer diameter of the inner molded portion 64 is too large, outer diameter OD_{IMP}, then necessarily the resulting outer diameter of the outer molded portion 66 (i.e., of the outer surface 96 of outer molded portion), outer diameter OD_{OMP}, will also increase, which can lead to having to reduce the height H_{ST} of the spline teeth 98 in order to keep the overall outer diameter OD_{SC} of the syringe cap 60 below a specified maximum diameter. Accordingly, in order to maintain desired thicknesses for the inner and outer molded portions 64, 66 while still enabling the formation of spline teeth 98 of a desired height H_{ST}, aspects of the disclosure are directed to molding the inner molded portion 64 in a manner that maintains the outer diameter OD_{IMP} thereof, as well as a thickness of the inner molded portion 64, thickness T_{IMP}, at desired levels, while decreasing an inner diameter ID_{IMP} of the inner molded portion 64 (i.e., a diameter of the inner surface 94 of the inner molded portion 64 at second end 70). According to embodiments, material is added to the inner surface 94 during the molding operation to obtain an inner molded portion 64 of desired thickness T_{IMP}, with the outer diameter OD_{IMP} of the inner molded portion 64 being formed to approximately 9.75 mm, +/−0.25 mm. By maintaining the outer diameter OD_{IMP} of the inner molded portion 64 at/below a desired value, the outer diameter OD_{IMP} of the outer molded portion 66 can also be maintained at/below a desired value, such that spline teeth 98 of sufficient height H_{ST} can be formed on the outer molded portion 66, thereby allowing for an efficient transfer of screwing torque to the syringe cap 60 via the spline teeth 98.

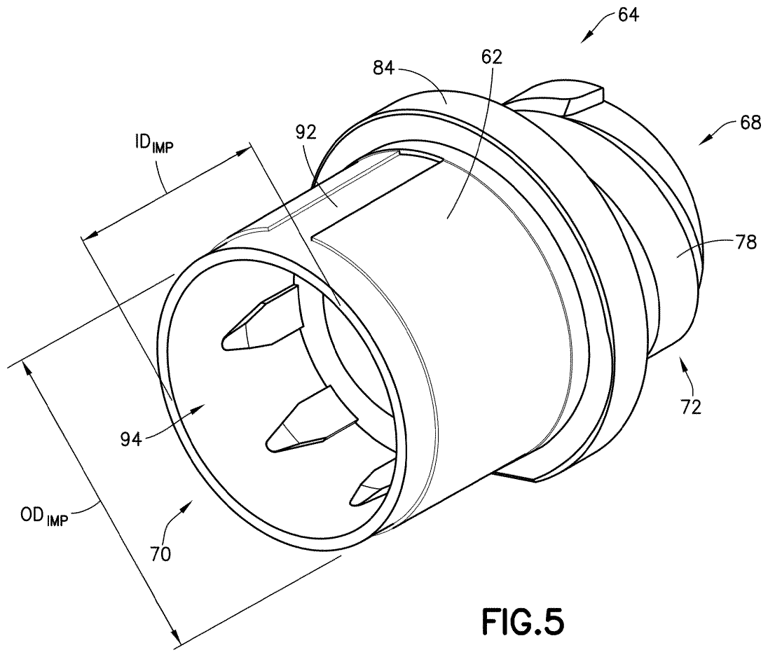

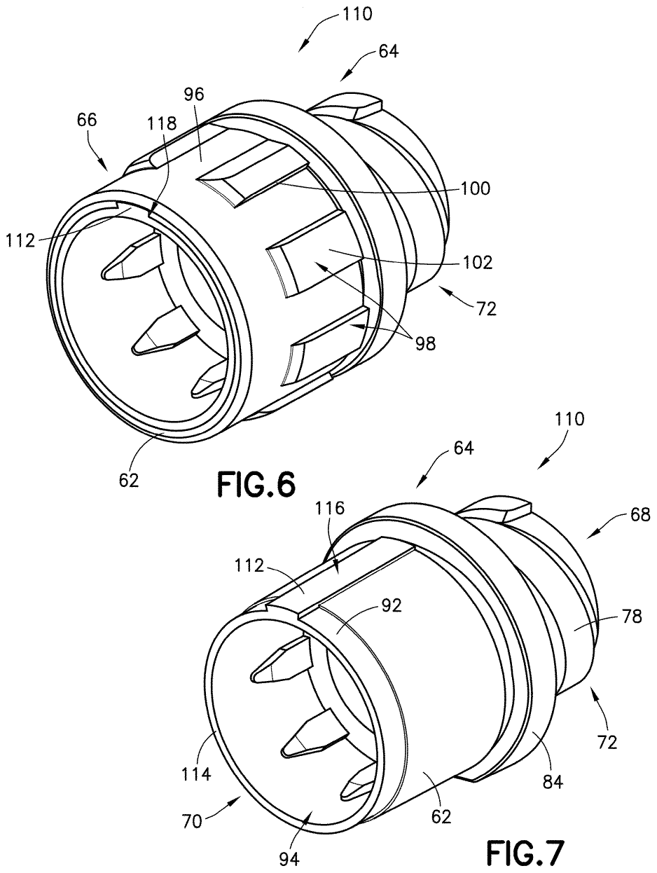

Referring now to FIGS. 6 and 7, a syringe cap 110 is illustrated in accordance with an additional aspect or embodiment of the disclosure, with it recognized that features of the cap 110 can be combined with features of the syringe cap 60 shown and described in FIGS. 3-5. The syringe cap 110 of FIGS. 6 and 7 may be structured substantially the same as the cap 60 of FIGS. 3-5, and thus like components are numbered the same, but the syringe cap 110 includes a rib 112 formed on the inner molded portion 64, as will be explained in greater detail below.

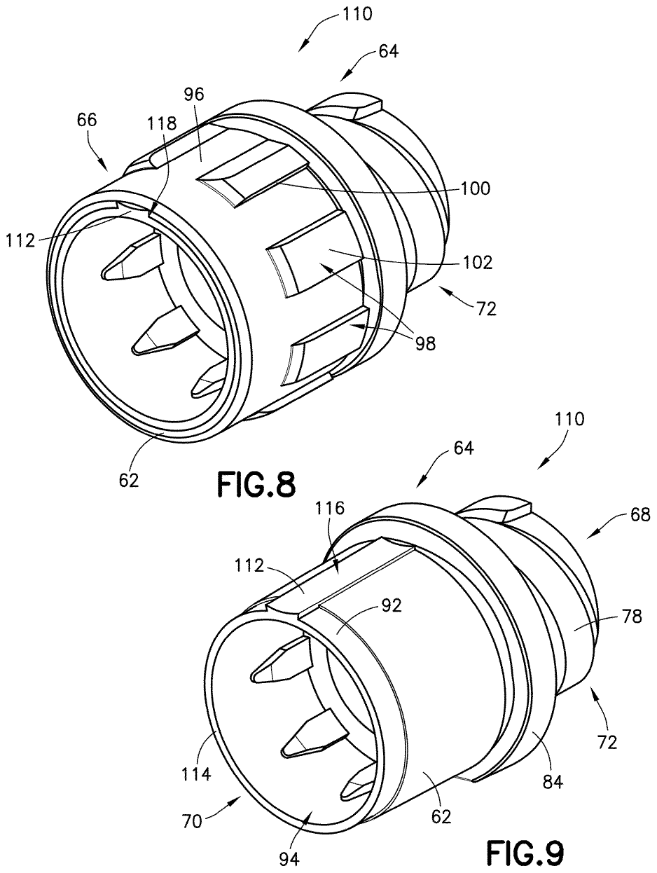

As shown best in FIG. 7, the second end 70 of inner molded portion 64 of syringe cap 110 includes a continuous sidewall 90 that provides a generally cylindrical outer surface 92 on the second end 70; however, a rib 112 is formed on the cylindrical outer surface 92 and extends radially outward therefrom. In some embodiments, the rib 112 extends longitudinally along the inner molded portion 64 from an edge 114 of the second end 70 to the annular flange 84. According to some aspects of the disclosure, and as shown in FIGS. 6 and 7, the rib 112 is formed such that a radially outward facing surface 116 thereof is generally flat. According to other aspects of the disclosure, and as shown in FIGS. 8 and 9, the rib 112 is formed such that the radially outward facing surface 116 thereof has a concave shape.

In accordance with some aspects of the disclosure, formation of the rib 112 on the outer surface 92 of the second end 70 of inner molded portion 64 provides an angular locator relative to which the RFID tag 62 may be applied/set on the inner molded portion 64. That is, in applying the RFID tag 62 to the inner molded portion 64, the RFID tag 62 may be positioned relative to the rib 112 such that the RFID tag 62 is wrapped about a majority of the cylindrical outer surface 92 of the second end 70, without being applied over or overlapping the rib 112. The rib 112 thus enables a simple and efficient angular locator relative to which the RFID tag 62 can be positioned during fabrication of the syringe cap 110.

With the rib 112 configured as shown in FIGS. 6 and 7 and/or as shown in FIGS. 8 and 9, the second molding process that forms outer molded portion 66 may be performed such that the outer molded portion 66 is formed over the inner molded portion 64, including over the rib 112. Resulting from such an application of the outer molded portion 66 over the inner molded portion 64 and over the rib 112, the rib 112 provides an interference between the inner molded portion 64 and the outer molded portion 66 that aids in retaining the outer molded portion 66 to the inner molded portion 64. That is, the rib 112 functions as a spline between the inner molded portion 64 and the outer molded portion 66 due to the rib 112 extending radially outward from a remainder of the outer surface 92 of the second end 70 of inner molded portion 64 and into a gap 118 formed in the outer molded portion 66 about the rib 112—with the rib 112 thus resisting shearing forces between the outer molded portion 66 and the inner molded portion 64 that may result from a screwing torque being applied to the outer molded portion 66 (by a twisting tool). Additionally, the rib 112 improves internal adhesion between the inner molded portion 64 and the outer molded portion 66 due to the molded polymeric material of the rib 112 (of the inner molded portion 64) being in a direct contact with molten polymeric material applied during molding of the outer molded portion 66—with a weld of the same material being created at the contact surface (i.e., surface 116) between the rib 112 and the outer molded portion 66. This adhesion between the rib 112 of inner molded portion 64 and the outer molded portion 66 is stronger than adhesion would be between inner and outer surfaces of the RFID tag 62 and the inner and outer molded portions 64, 66, and negates the need for performing an expensive double-sided lamination of the RFID tag 62 (i.e., adding thin layers of molded material thereto, separate from the first/second molding operations) necessary to improve RFID tag wettability, thereby adding to the integrity of the syringe cap 110 and reducing a cost thereof.

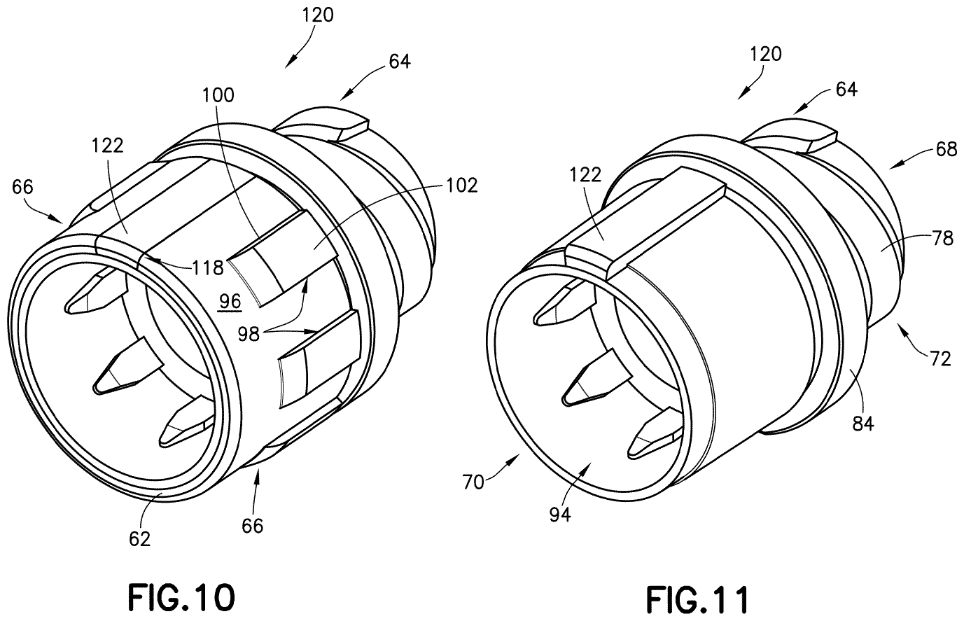

Referring now to FIGS. 10 and 11, a syringe cap 120 is illustrated in accordance with an additional aspect or embodiment of the disclosure, with the syringe cap 120 including a rib 122 that is structured differently from the rib 112 of syringe cap 110 in FIGS. 6 and 7 and/or FIGS. 8 and 9. In the syringe cap 120 of FIGS. 10 and 11, the rib 122 formed on the inner molded portion 64 has an increased thickness/height as compared to the rib 112 of syringe cap 110, such that in molding the outer molded portion 66 over inner molded portion 64, the outer molded portion 66 is formed over the inner molded portion 64 except for the rib 122, whereby the rib 122 is not covered by the outer molded portion 66. Instead, the rib 122 is configured to have a thickness/height whereby, upon molding the outer molded portion 66 over inner molded portion 64 (except for the rib 122), the radially outward facing surface 116 of the rib 122 is flush with the outer surface 96 of outer molded portion 66.

As previously described, the rib 122 provides an interference between the inner molded portion 64 and the outer molded portion 66 that aids in retaining the outer molded portion 66 to the inner molded portion 64. That is, the rib 122 functions as a spline between the inner molded portion 64 and the outer molded portion 66 due to the rib 122 extending radially outward from a remainder of the outer surface 92 of the second end 70 of inner molded portion 64 and into a gap 118 formed in the outer molded portion 66 about the rib 122—with the rib 122 thus resisting shearing forces between the outer molded portion 66 and the inner molded portion 64 that may result from a screwing torque being applied to the outer molded portion 66 (by a twisting tool). Additionally, the rib 122 improves internal adhesion between the inner molded portion 64 and the outer molded portion 66 due to the molded polymeric material of the rib 122 (of the inner molded portion 64) being in a direct contact with molten polymeric material applied during molding of the outer molded portion 66—with a weld of the same material being created between the contact surfaces (i.e., side surfaces) of the rib 122 and the outer molded portion 66.

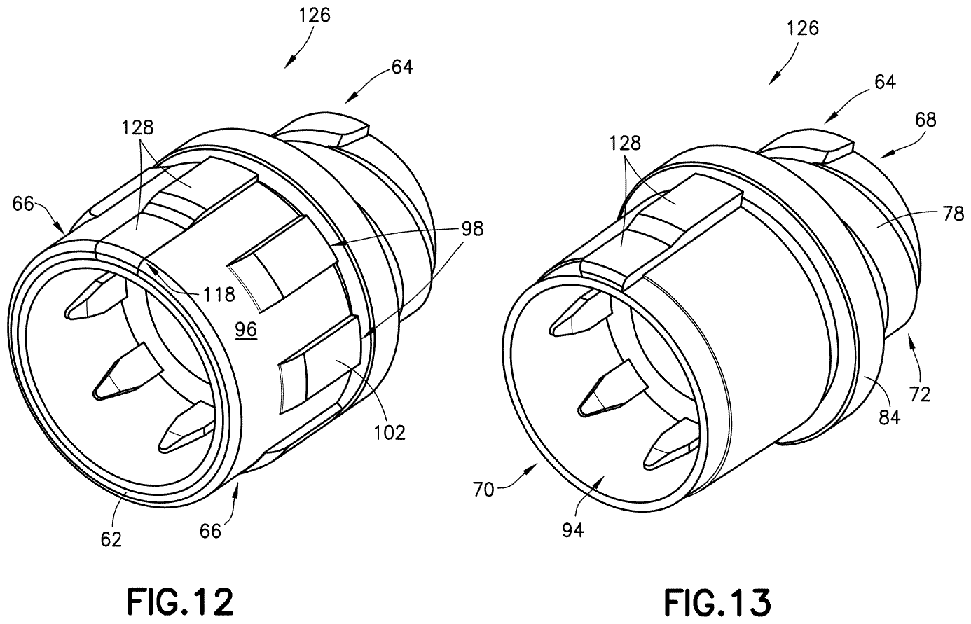

Referring now to FIGS. 12 and 13, a syringe cap 126 is illustrated in accordance with an additional aspect or embodiment of the disclosure, with the syringe cap 126 including a rib 128 that is structured differently from the rib 112 of cap 110 in FIGS. 6 and 7 (or FIGS. 8 and 9) and the rib 122 of cap 120 in FIGS. 10 and 11. In the syringe cap 126 of FIGS. 12 and 13, the rib 128 formed on the inner molded portion 64 has a still further increased thickness/height as compared to the rib 122 of syringe cap 120, such that not only is the rib 128 not covered by the outer molded portion 66 upon molding of the outer molded portion 66 over inner molded portion 64, but the rib 128 has a thickness/height such that at least a portion of the rib 128 extends radially outward past the outer surface 96 of the outer molded portion 66 and to a height equal to the height H_{ST} of the plurality of spline teeth 98 on the outer molded portion 66.

With the rib 128 structured as described above, the rib 128 both provides for interference and adhesion between the inner molded portion 64 and the outer molded portion 66 and also functions as a spline tooth on the syringe cap 126. First, and as described above, the rib 128 functions as a spline between the inner molded portion 64 and the outer molded portion 66 that resists shearing forces between the outer molded portion 66 and the inner molded portion 64 that 16 may result from a screwing torque being applied to the outer molded portion 66 (by a twisting tool), while also improving internal adhesion between the inner molded portion 64 and the outer molded portion 66 due to the molded polymeric material of the rib 128 (of the inner molded portion 64) being in a direct contact with molten polymeric material applied during molding of the outer molded portion 66. Second, resulting from the rib 128 extending radially outward past the outer surface 96 of the outer molded portion 66 and to a height equal to the height H_{ST} of the plurality of spline teeth 98 on the outer molded portion 66, the rib 128 is configured to engage with a twisting tool and provide for the transmission of a screwing torque from the tool to the syringe cap 126 for securing thereof to the syringe barrel 12 (i.e., enabling a threaded engagement of the female connector 72 of cap to the male connector of syringe barrel 12).

Beneficially, embodiments of the invention thus provide a syringe cap with an integrated RFID tag and torque transmission features formed thereon. The syringe cap is formed as a co-molded cap with the RFID tag embedded therein between distinct inner and outer molded portions or layers of the cap. The syringe cap includes internal and external features thereon that enable torque applied to the cap, such as via a twisting tool engageable with the cap, to be transmitted through the cap, so as to provide for twisting engagement of the cap with an associated connection on a syringe barrel. Internal and external dimensions of the syringe cap are controlled so as to properly embed the RFID tag therein, while still providing spline teeth of sufficient height on the syringe cap that provide for an efficient transmission of external torque to the cap. Additionally, a rib feature may be included in the syringe cap that provides improved cohesion between distinct inner and outer molded portions or layers of the cap and that resists shear forces within the cap, so as to provide for an efficient transmission of internal torque within the cap.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

1. A syringe cap for use with a syringe, the syringe cap comprising:
an inner molded portion comprising a first end and a second end and defining an internal cavity, the first end comprising an open end and including a female connector formed thereon and the second end comprising a closed end;
a radio frequency identification (RFID) element positioned over the inner molded portion at the second end, so as to extend circumferentially about at least a portion of the second end; and
an outer molded portion formed over the inner molded portion and over the RFID element at the second end, so as to encase the RFID element between the inner molded portion and the outer molded portion, the outer molded portion comprising an outer surface including a plurality of spline teeth spaced circumferentially thereabout and extending radially outward from the outer surface;
wherein the plurality of spline teeth have a height of 0.400 mm or more extending radially out from the outer surface, and
wherein the RFID element comprises a flat RFID inlay that conforms to a cylindrical outer surface of the second end of the inner molded portion.

2. The syringe cap of claim 1, wherein each of the plurality of spline teeth extend out between 0.400 and 0.750 mm from the outer surface.

3. The syringe cap of claim 1, wherein the second end of the inner molded portion comprises a circumferential outer surface, and wherein an outer diameter of the second end is approximately 9.75 mm.

4. The syringe cap of claim 1, wherein the female connector has a threaded outer surface configured to engage a male connector, and wherein the height of the plurality of spline teeth enables engagement of the second end with a twisting tool, for threadingly engaging the threaded outer surface of the female connector to the male connector.

5. The syringe cap of claim 1, wherein the inner molded portion comprises an annular flange formed distally from the threaded outer surface, between the first end and the second end.

6. The syringe cap of claim 1, wherein the second end of the inner molded portion comprises a rib formed on a cylindrical outer surface thereof, the rib extending radially outward from the cylindrical outer surface.

7. The syringe cap of claim 6, wherein the rib comprises an angular locator, which the RFID element is positioned relative to, with the RFID element positioned over the cylindrical outer surface of the second end so as to not overlap the rib.

8. The syringe cap of claim 6, wherein the outer molded portion is formed over the inner molded portion, including over the rib, with the rib forming an interference between the inner molded portion and the outer molded portion.

9. The syringe cap of claim 8, wherein a radially outward facing surface of the rib has a concave shape.

10. The syringe cap of claim 6, wherein the outer molded portion is formed over the inner molded portion, except for the rib, such that the rib is not covered by the outer molded portion, and wherein a radially outward facing surface of the rib is flush with the outer surface of outer molded portion.

11. The syringe cap of claim 6, wherein the outer molded portion is formed over the inner molded portion, except for the rib, such that the rib is not covered by the outer molded portion, and wherein a portion of rib extends radially outward past the outer surface of the outer molded portion to a height equal to the height of the plurality of spline teeth.

12. A syringe, comprising:
a syringe barrel comprising a proximal end and a distal end and defining a chamber, the syringe barrel having an opening at the proximal end;
a plunger assembly inserted through the opening and axially movable within the chamber of the syringe barrel, the plunger assembly including:
a plunger rod comprising an elongated body and extending between a proximal end and a distal end; and
a stopper attached to the distal end of plunger rod and positioned within the barrel chamber; and
a syringe cap, the syringe cap comprising:
an inner molded portion comprising a first end and a second end and defining an internal cavity, the first end comprising an open end and including a female connector formed thereon and the second end comprising a closed end,
a radio frequency identification (RFID) element positioned over the inner molded portion at the second end, so as to extend circumferentially about at least a portion of the second end, and
an outer molded portion formed over the inner molded portion and over the RFID element at the second end, so as to encase the RFID element between the inner molded portion and the outer molded portion, the outer molded portion comprising an outer surface including a plurality of spline teeth spaced circumferentially thereabout and extending radially outward from the outer surface,
wherein the plurality of spline teeth have a height of 0.400 mm or more extending radially out from the outer surface,
wherein the female connector of the inner molded portion is coupled to a male connector provided at the distal end of the syringe barrel, and
wherein the RFID element comprises a flat RFID inlay that conforms to a cylindrical outer surface of the second end of the inner molded portion.

13. A method of forming a co-molded syringe cap, the method comprising:
molding an inner cap portion comprising a first end and a second end and defining an internal cavity, the first end comprising an open end and including a female connector formed thereon and the second end comprising a closed end;
setting a radio frequency identification (RFID) element onto the inner molded portion at the second end, with the RFID element set so as to extend circumferentially about at least a portion of the second end; and
molding an outer cap portion over the inner molded portion and over the RFID element at the second end, so as to encase the RFID element between the inner cap portion and the outer cap portion, the outer cap portion comprising an outer surface including a plurality of spline teeth spaced circumferentially thereabout and extending radially outward from the outer surface;
wherein the plurality of spline teeth have a height of 0.400 mm or more extending radially out from the outer surface, and
wherein the RFID element comprises a flat RFID inlay that conforms to a cylindrical outer surface of the second end of the inner molded portion.

14. The method of claim 13, wherein in molding the outer cap portion, each of the plurality of spline teeth is formed to extend out between 0.400 and 0.750 mm from the outer surface.

15. (canceled)

16. The method of claim 13, wherein molding the inner cap portion comprises forming a rib on a cylindrical outer surface of the inner cap portion, the rib extending radially outward from the cylindrical outer surface.

17. (canceled)

18. (canceled)

19. (canceled)

20. A syringe cap for use with a syringe, the syringe cap comprising:
an inner molded portion comprising a first end and a second end and defining an internal cavity, the first end comprising an open end and including a female connector formed thereon and the second end comprising a closed end;
a radio frequency identification (RFID) element positioned over the inner molded portion at the second end, so as to extend circumferentially about at least a portion of the second end; and
an outer molded portion formed over the inner molded portion and over the RFID element at the second end, so as to encase the RFID element between the inner molded portion and the outer molded portion, the outer molded portion comprising an outer surface including a plurality of spline teeth spaced circumferentially thereabout and extending radially outward from the outer surface;
wherein the second end of the inner molded portion comprises a rib formed on a cylindrical outer surface thereof, the rib extending radially outward from the cylindrical outer surface, to form an interference between the inner molded portion and the outer molded portion, and
wherein the RFID element comprises a flat RFID inlay that conforms to a cylindrical outer surface of the second end of the inner molded portion.

21. The syringe cap of claim 20, wherein the rib comprises an angular locator, which the RFID element is positioned relative to, with the RFID element positioned over the cylindrical outer surface of the second end so as to not overlap the rib.

22. The syringe cap of claim 20, wherein the outer molded portion is formed over the inner molded portion, including over the rib.

23. The syringe cap of claim 20, wherein the outer molded portion is formed over the inner molded portion, except for the rib, such that the rib is not covered by the outer molded portion, and wherein a radially outward facing surface of the rib is flush with the outer surface of outer molded portion.

24. The syringe cap of claim 20, wherein the outer molded portion is formed over the inner molded portion, except for the rib, such that the rib is not covered by the outer molded portion, and wherein a portion of rib extends radially outward past the outer surface of the outer molded portion to a height equal to the height of the plurality of spline teeth.
